(19) United States
(12) Patent Application Publication
(10) Pub. No.: US 2009/0042801 A1
(43) Pub. Date: Feb. 12, 2009

(54) Method for inhibiting infection and reproduction of influenza type A WSN virus

(75) Inventors: Chuang Chun Chiueh, Taipei (TW); Shin Ru Shih, Taipei (TW)

Correspondence Address: BACON & THOMAS, PLLC, 625 SLATERS LANE, FOURTH FLOOR, ALEXANDRIA, VA, 22314-1176, US

(73) Assignee: Industrial Technology Research Institute, Hsinchu (TW)

(21) Appl. No.: 12/285,349

(22) Filed: Oct. 2, 2008

Related U.S. Application Data
(63) Continuation-in-part of application No. 11/128,189, filed on May 13, 2005.

(30) Foreign Application Priority Data
Sep. 21, 2004 (TW) 93128553

(51) Int. Cl. A61K 38/00; A61P 31/12

(52) U.S. Cl. 514/12

(57) ABSTRACT

The present invention relates to a method for inhibiting infection and reproduction of influenza type A WSN virus, which comprises providing an effective amount of a pharmaceutical composition; and contacting said composition with said influenza type A WSN virus, wherein said pharmaceutical composition contains C-phycocyanin (C-PC), allophycocyanin (APC), and spirulina growth factor (SGF). The present invention also provides a method for extracting said pharmaceutical composition, comprising the steps of: (a) adding hypotonic buffer solution to organic blue-green algae powder and mixing thoroughly; (b) incubating the mixture below room temperature overnight; (c) separating and purifying the mixture by a centrifuge; (d) collecting the suspending supernatant and detecting it by a spectrometer to determine ingredients and content; and (e) spray drying the supernatant; characterized in which low-temperature extraction is employed to maintain the bioactivity and nutrients of the pharmaceutical composition.

### CROSS REFERENCES TO THE RELATED APPLICATIONS

This is a Continuation-in-part of U.S. application Ser. No. 11/128,189, filed May 13, 2005, now pending.

### BACKGROUND OF THE INVENTION

1. Field of the Invention

The present invention discloses a method for inhibiting infection and reproduction of influenza type A WSN virus that can be applied to the prevention and treatment of influenza caused by said influenza type A WSN virus.

2. Description of Related Art

Influenza is a infectious disease caused by a filterable virus which is a RNA virus and classified as a member of the Family Orthomyxoviridae. According to different serum immune responses, influenza viruses are classified into type A, B and C. Flu happens commonly in winter and in early spring; virus infiltrates the lung via mouth and/or nose to cause infection, in particular in congested area or public place. According to historical records, influenza virus has caused once epidemical feverish respiratory disease every three to four years. Worldwide flu epidemic occurs once approximately every ten years. In the 1918 influenza pandemic, at least 22 million people died. Influenza virus is estimated to cause the worldwide death of 60 million people. Besides claiming lives, influenza virus also easily leads to serious complications in children or elderly people and contributes to enormously economic loss. Thus influenza virus is one of the key topics of research worldwide.

Blue-green algae has been existing on earth for billions of years. The essences it contains such as phycocyanin, spirulina and sulfur polysaccharides are found effective against microorganisms. The studies of Harvard Medical School, University of Illinois, Toyama Medical and Pharmaceutical University, and in Germany, Russia and Mexico respectively demonstrate that blue-green algae extract has significant inhibitory effect against Picomaviridae as well as Family Paramyxoviridae, including measles virus, mumps virus, herpes virus and HIV.

U.S. Pat. No. 4,851,339 is related to a composition for nutrition purposes comprising C-phycocyanin and allophycoccyanin which has been extracted from Spirulina and US Patent No. 2002/0009479 is related to a food composition containing viable algae, while the present invention is related to a method for inhibiting infection and reproduction of influenza type A WSN virus. Hence, the present invention is different from the two US patents.

Disclosed in U.S. Pat. No. 6,346,408 and other literature (master's dissertation of Tsai Kun-nan from Chang Gung University Basic Medicine Research Institute), a group of water-soluble proteins—phycocyanin uniquely found in algae are capable of inhibiting the reproduction of enterovirus and influenza virus which lead to cytopathic effect through two mechanisms: 1. preventing infection; and 2. delaying virus reproduction in infected cells. It is found that at the concentration of 0.3 μM or higher, phycocyanin effectively prevents viral infection. Cellular experiments clearly show that phycocyanin at such concentration has no cytotoxicity, but effectively protects cells from the invasion of virus and pathological symptoms. Phycocyanin at an effective concentration of 0.04 μM or higher can also prevent the infection of influenza virus. Adding phycocyanin before the viruses react with cells gives the cells better protection.

Because virus infection leads to cytopathic effect, currently vaccines and anti-viral drugs are used for prevention and treatment against virus. Although vaccines are effective, they are not suitable for all cases. On the other hand, there has not been significant breakthrough in anti-viral drugs development. Therefore, it is important to develop a pharmaceutical composition that can effectively inhibit the reproduction of influenza virus.

### SUMMARY OF THE INVENTION

In light of the potential of blue-green algae extract, the present invention provides a method for inhibiting the infection and reproduction of influenza type A WSN virus, which comprises providing an effective amount of a pharmaceutical composition; and contacting said pharmaceutical composition with said influenza type A WSN virus, wherein said pharmaceutical composition contains C-phycocyanin (C-PC), allophycocyanin (APC), and spirulina growth factor (SGF).

Another object of the present invention is to provide a method for extracting the aforesaid pharmaceutical composition, comprising the steps of: (a) adding hypotonic buffer solution to organic blue-green algae powder and mixing soundly; (b) incubating the mixture below room temperature overnight; (c) separating and purifying the mixture by a centrifuge; (d) collecting the suspending supernatant and detecting by a spectrometer to determine ingredients and content; and (e) spray drying the supernatant; characterized in which low-temperature extraction at 0° C.˜18° C. is employed to maintain the bioactivity and nutrients of the pharmaceutical composition.

### BRIEF DESCRIPTION OF THE DRAWINGS

FIG. 1 illustrates the therapeutic effect of the pharmaceutical composition according to the present invention.

FIG. 2 illustrates the preventive effect of the pharmaceutical composition according to the present invention, wherein the mortality of Tamiflu-A is 2/6 (33%) and the mortality of virus only is 3/6 (50%).

### DETAILED DESCRIPTION OF THE INVENTION

In substantial applications, the pharmaceutical composition may further selectively contain a pharmaceutically acceptable carrier, adjuvant, excipient or additive in addition to an effective amount of C-phycocyanin (C-PC), allophycocyanin (APC), and spirulina growth factor (SGF).

Said “carrier” herein may contain inert component which does not react substantively with other ingredients. Applicable drug formulation techniques may refer to standard drug formulation techniques as described in Remington's Pharmaceutical Sciences by Mack Publishing Company, Easton, Pa. Suitable drug carrier includes but not limited to sterilized water, normal saline, bacteriostatic saline (containing approximately 0.9% phenyl alcohol), phosphate-buffered saline, Hank's solution, lactated Ringer's solution and other commonly used pharmaceutical carriers.

Said “excipient” herein may have multiple functions and purposes, for example, adding disintegrating agent to disintegrate lozenges into tiny granules in the gastrointestinal tract and facilitate its absorption in the process of making oral lozenge, or adding colorant to make it more pleasing and so on. There are other suitable excipients respectively for non-oral formulations, such as injection, suspension, ointment, suppository and spray. Suitable excipients include but not limited to lactose, mannitol, dextran, glucose, glutamic acid, gelatin, sorbitol, trehalose, sucrose, xylitol, starch, microcrystalline cellulose, methyl cellulose, arabic gum or combinations. The utilizing of excipients is common knowledge in this field.

The pharmaceutical composition herein is not limited to the use of C-phycocyanin (C-PC), allophycocyanin (APC) and spirulina growth factor (SGF) mixture. It can be used in combination with other supplements or drugs known to prevent flu or alleviate the symptoms of flu, including but not limited to aspirin, p-acetyl sodium, pseudoephedrine hydrochloride, ephedrine hydrochloride, chlor-trimeton, benzhydramine, dextromethophan hydrobromide, clopamide, indigowood root and Vitamin C.

The “effective amount” means the dose of the compound that will produce beneficial result in the recipient or expected activity in vivo or in vitro. Taking the example of flu, clinical benefits compared to subjects not receiving the treatment include alleviation of symptoms, mitigation of discomfort, shortening the duration of illness, and accelerated healing. The precise dosage to different subjects will be determined by the disease type, severity or symptoms of disease and individual conditions, such as the health state, age, gender, body weight and drug tolerance of the recipient. People familiar with the field may decide dosage based on the factors described above or other factors.

Based on needs, the pharmaceutical composition according to the present invention may be made into powder, spray, granule, liquid, gel or paste by the process that is known to one skilled in the art. The formulation supplied can modify with the administration route and different target signs or symptoms.

The pharmaceutical composition according to the present invention can be administered through suitable routes, for example, but not limited to oral capsule, suspension or tablet, or parenteral pathway. Parenteral administration includes, for example, intramuscular, intravenous, hypodermic or intraperitoneal injection. The composition may also be taken orally (e.g. contained in food), or through local injection, inhalation (e.g. intrabronchial, intranasal, inhalation through mouth or intranasal drip), or rectally. The method of administration should be determined by the target disease and its signs or symptoms.

The solid preparation of the pharmaceutical composition, if taken orally, may take the form of, for example, but not limited to capsule, lozenge, sugar-coated tablet, pill, powder and granules, or be prepared with a coating (e.g. enteric coating) by the process that is known to one skilled in the art. It can also be prepared to regulate the release of active composition, for example, sustaining or extending release of active ingredients. The liquid preparation of the pharmaceutical composition, if taken orally, may take the form of solution, emulsion, suspension, syrup and elixir.

The pharmaceutical composition herein may be provided in the form of food, beverage, drug, reagent or nutritional supplement.

The pharmaceutical composition herein is applicable to inhibiting the infection and reproduction of influenza viruses, in particular, but not limited to the infection and reproduction of influenza type A WSN virus.

The pharmaceutical composition herein is applicable to the prevention and/or treatment of all kinds of influenza, in particular to the prevention of all kinds of influenza.

The pharmaceutical composition herein is applicable to the prevention and/or treatment of all kinds of influenza, in particular, but no limited to the prevention and/or treatment of influenza caused by influenza type A WSN virus.

Prior art techniques use hot water to extract organic blue green algae, but high temperature easily causes the degradation or degeneration of its protein nutrients and leads to the loss of activity. To address this problem, it is another object of the present invention to provide a method for extracting the aforementioned pharmaceutical composition, comprising the steps of: (a) adding hypotonic buffer solution to organic blue-green algae powder and mixing thoroughly; (b) incubating the mixture below room temperature overnight; (c) separating and purifying by a centrifuge; (d) collecting the suspending supernatant and detecting by a spectrometer to determine ingredients and content; and (e) spray drying the supernatant; characterized in which low-temperature extraction is employed to maintain the bioactivity and nutrients of the pharmaceutical composition. The volume of said hypotonic solution is preferably ten times that of organic blue-green algae powder. The entire process is preferably undertaken under 0˜18° C., and optimally under 4° C. The use of hypotonic solution creates an osmotic pressure gradient to render cell wall and cell membrane more permeable, which helps the release of active ingredients. In comparison with prior art extraction techniques, the method provided herein has the following advantages: 1. The bioactivity of the active ingredients is maintained; and 2. There is no need to use glass beads to smash the algae, hence preventing the contamination of glass fragments and loss of bioactivity caused by heat generated in the process.

The advantages of the present invention are further depicted with the illustration of examples.

### EXAMPLES

Below are examples using the pharmaceutical composition for inhibiting influenza virus infection and reproduction provided herein, but the descriptions made in the examples should not be construed as a limitation on the actual application of the present invention. All modifications and alterations made by those familiar with the skill without departing from the spirits of the invention and appended claims shall remain within the protected scope and claims of the invention.

### Example 1

### The Pharmaceutical Composition According to the Invention Can Inhibit the Reproduction of Type A Influenza Virus in ICR Mice

### Materials

### Chemicals

1. Mixture of C-phycocyanin (C-PC), allophycocyanin (APC), and spirulina growth factor (SGF) extracted under low temperature.

2. Anesthetics—Ketamine

3. Type A influenza virus—Influenza WSN virus at concentrations of 1500 pfu, 150 pfu and 15 pfu.

### Mice

Parental ICR mice introduced from National Experimental Animal Center were bred and reared for four weeks old. 8 female ICR mice were arranged into one cage which designed as a group. The room temperature was kept under 16˜18° C. and light cycle time was 12 hours.

### Experimental Steps

Five weeks old female ICR mice were divided into seven groups, which were:

Group I—Gavaged with 1500 pfu type A influenza virus and the prepared pharmaceutical composition.

Group II—Gavaged with 150 pfu type A influenza virus and the prepared pharmaceutical composition.

Group III—Gavaged with 15 pfu type A influenza virus and the prepared pharmaceutical composition.

Control group A—Gavaged with the prepared pharmaceutical composition without virus.

Control group B—Gavaged with 1500 pfu type A influenza virus and phosphate buffer solution (PBS).

Control group C—Gavaged with 150 pfu type A influenza virus and phosphate buffer solution.

Control group D—Gavaged with 15 pfu type A influenza virus and phosphate buffer solution.

Mice in the experimental groups were anesthetized by Ketamine (1 mg per mouse) and immediately gavaged with type A influenza virus (Influenza WSN virus at 1500 pfu, 150 pfu, and 15 pfu). The control groups went through the same procedure, but received no virus and PBS only.

To observe the ability of the pharmaceutical composition to inhibit virus activity, mice in the experimental groups were given the pharmaceutical composition once a day for six days consecutively after they first received the virus for 6 hours later. The dose given was 18 mg/kg. The mice were reared for eight days. Daily observation of weight change and mortality was carried out.

The results as shown in Table 1 and Table 2 demonstrate that mice in Control group B, C and D that were given only virus but no pharmaceutical composition died respectively on day 4 (1500 pfu), day 5 (150 pfu) and day 7 (15 pfu) after viral infection. But mice in the experimental groups that were given both virus and pharmaceutical composition survived. Although those mice showed mild symptom of illness accompanied with weight loss in the beginning, they all recovered gradually and gained weight again. The body weight of mice in the control group that were given only the pharmaceutical composition without virus increased with time.

As shown in the result of the experiment, the pharmaceutical composition according to the present invention can totally prevent the death of mice infected with type A influenza virus and can be used to inhibit the activity of such virus.

TABLE 1
Weight Change and Mortality Record of Mice in Control Groups

| | Control | Control | Control | Control |
|---|---|---|---|---|
| | group A | group B | group C | group D |
| Virus | — | 1500 pfu | 150 pfu | 15 pfu |
| Pharmaceutical | + | − | − | − |
| composition | | | | |
| Weight (g) | | | | |
| Day 0 | 19.10 | 19.21 | 19.23 | 19.08 |
| Day 1 | 19.53↑ | 18.01 | 18.57 | 19.01 |
| Day 2 | 20.57↑ | 16.51 | 17.11 | 18.35 |
| Day 3 | 21.50↑ | Dead | 16.11 | 17.31 |
| Day 4 | 22.67↑ | | Dead | 17.34 |
| Day 5 | 23.50↑ | | | 16.31 |
| Day 6 | 24.63↑ | | | Dead |
| Day 7 | 25.73↑ | | | |
| Day 8 | Survived | | | |

TABLE 2
Weight Change and Mortality Record of
Mice in Experimental Groups

| | Control group I | Control group II | Control group III |
|---|---|---|---|
| Virus | 1500 pfu | 150 pfu | 15 pfu |
| Pharmaceutical | + | + | + |
| composition | | | |
| Weight (g) | | | |
| Day 0 | 19.16 | 19.25 | 19.66 |
| Day 1 | 18.54 | 18.51 | 19.11 |
| Day 2 | 18.91 | 18.98 | 19.49 |
| Day 3 | 18.91 | 19.26↑ | 18.85 |
| Day 4 | 19.34↑ | 19.63↑ | 19.13↑ |
| Day 5 | 19.76↑ | 19.93↑ | 19.16↑ |
| Day 6 | 19.79↑ | 20.14↑ | 19.66↑ |
| Day 7 | 19.81↑ | 20.36↑ | 20.01↑ |
| Day 8 | Survived | Survived | Survived |

### Example 2

### The Pharmaceutical Composition According to the Invention Can Inhibit the Reproduction of Type A Influenza Virus in Balb/c Mice

Experiment animal: Balb/c mice five weeks old were used for this experiment.

Experiment virus: Type A influenza virus—Influenza WSN virus at concentration of 3.4×10⁴ pfu

### Administration Dose:

1. The pharmaceutical composition according to the present invention at the dose of 25 mg/kg every day

2. Tamiflu as a positive control at the dose of 10 mg/kg every day

### Administration Method:

The Balb/c mice were given the pharmaceutical composition according to the present invention once before the virus is administrated to them. After the mice were infected, they received the pharmaceutical composition once, and then received the pharmaceutical composition twice a day for five days consecutively. The group used Tamiflu as a drug of flu treatment went through the same procedure as above described.

### Observation Item:

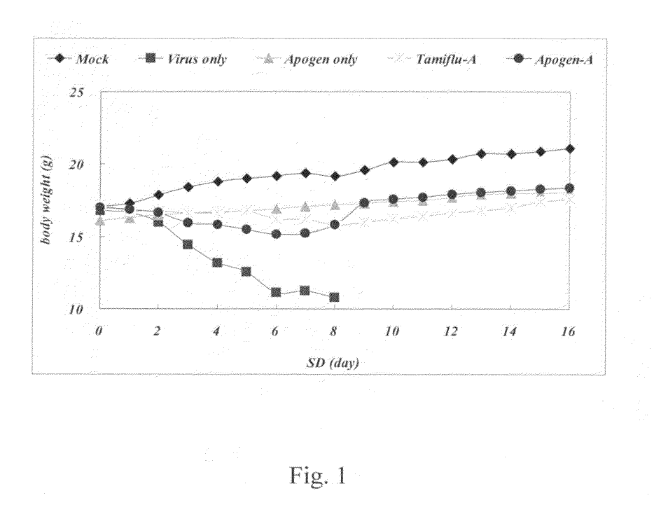

Daily observation of weight change and mortality was carried out for sixteen days consecutively after the mice were infected. The results were shown in FIG. 1.

The experimental result as shown in FIG. 1 exhibits the body weight of mice treated with the pharmaceutical composition according to the present invention were increased with time. Accordingly, the pharmaceutical composition has therapeutic effect for influenza caused by influenza type A WSN virus.

### Example 3

### The Pharmaceutical Composition According to the Invention Can Prevent the Reproduction of Type A Influenza Virus in Balb/c Mice

Experiment animal: Balb/c mice four weeks old were used for this experiment.

Experiment virus: Type A influenza virus—Influenza WSN virus at concentration of 3.4×10⁴ pfu

### Administration Dose:

1. The pharmaceutical composition according to the present invention at the dose of 100 mg/kg and 25 mg/kg every day

2. Tamiflu as a positive control at the dose of 10 mg/kg every day

### Administration Method:

The Balb/c mice were given the pharmaceutical composition according to the present invention twice a day for seven days consecutively before the virus is administrated to them. However, the mice received no the pharmaceutical composition if they were infected with the virus. The group used Tamiflu as a drug of flu treatment went through the same procedure as above described.

### Observation Item:

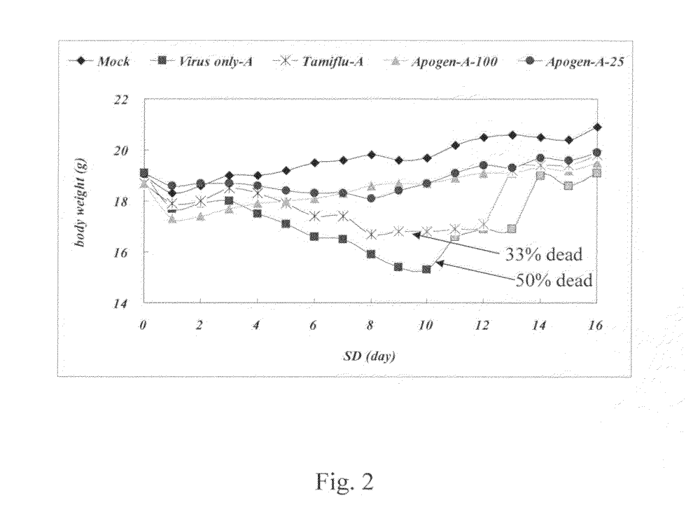

Daily observation of weight change and mortality was carried out for sixteen days consecutively after viral infection. The results were shown in FIG. 2.

The results as shown in FIG. 2 indicate that the pharmaceutical composition of present invention can prevent viral infection.

As shown in the results, experimental animals that were given the pharmaceutical composition beforehand were able to recover from the virus-induced symptoms, while control group animals which were not administered with the composition died, indicating its efficacy in inhibiting the reproduction of type A influenza virus and hence preventing viral infection. According to TW Patent Application No. 08911092 (corresponding U.S. Pat. No. 6,346,408) entitled “Method for inhibiting the cytopathic effect of enterovirus and influenza virus by using phycobilins”, treatment with allophycocyanin at the time of and after type A influenza virus infection could reach IC50. Moreover, referring the Table 3 and Table 4, they show the IC50 value of present invention is less than that of SGF+CPC and SGF+APC, and the LC50 (lethal concentration 50%) value of present invention is more than that of others, respectively. To sum up above, they indicate the inhibiting effect of present invention is superior to that of the composition of two components and the composition of present invention has less cytotoxicity (less side effects), respectively.

TABLE 3
The compared result of the IC50 value

| Composition | IC50 (μg/mL) |
|---|---|
| SGF | 538.5 |
| SGF + C-PC | 710 |
| SGF + APC | 622 |
| The present invention (C-PC + APC + SGF) | 68.40 |

TABLE 4
The compared result of the LC50 value

| | LC50 (μg/mL) |
|---|---|
| Composition | MDCK cell |
| APC | 43.58 |
| C-PC | 30.96 |
| SGF | >2000.00 |
| SGF + CPC | ~2000.00 |
| SGF + APC | ~2000.00 |
| The present invention (C-PC + APC + SGF) | >2500.00 |

The dosage of the pharmaceutical composition administered to mice in this experiment was 18 mg/kg, which produced different level of improvement on mice infected with different concentrations of type A influenza virus. It is well known to one skilled in the art that long-term use of pharmaceutical composition might produce dose-dependent phenomenon i.e. the higher the dosage used, the more pronounced the improvement is. Thus the dose of the pharmaceutical composition should be adjusted along with the duration and symptoms of the disease.

The pharmaceutical composition herein is also applicable to mammals other than mice. The conversion of effective dose as applied to different mammals is also known to one skilled in the art. For instance, the conversion of mice dosage to human dosage may use liver surface area or the coefficient of 1:100. That is, divide the mice dosage by said coefficient to obtain dosage for humans. As known to one skilled in the art, the precise dosage for individual recipient should be determined by the infectious level, severity, infectious type, and individual conditions, such as general health status, age, gender, body weight, and drug tolerance.

### OTHER EMBODIMENTS

All features disclosed herein may be combined in any form with other methods and replaced by other features with identical, equivalent or similar purpose. Thus except for the part that is specifically emphasized, all features disclosed herein constitute only one embodiment among the numerous equivalent or similar features.

All modifications and alterations to the descriptions disclosed herein made by those skilled in the art without departing from the spirits of the invention and appended claims shall remain within the protected scope and claims of the invention.

What is claimed is:

1. A method for inhibiting infection and reproduction of influenza type A WSN virus, comprising:
providing an effective amount of a pharmaceutical composition; and
contacting said pharmaceutical composition with said influenza type A WSN virus,
wherein said pharmaceutical composition contains C-phycocyanin, allophycocyanin, and spirulina growth factor.

2. The method according to claim 1, wherein said pharmaceutical composition further contains pharmaceutically acceptable carrier, adjuvant, excipient or additive.

3. The method according to claim 1, wherein said pharmaceutical composition exists in the form of powder, granule, liquid, gel or paste.

4. The method according to claim 1, wherein said pharmaceutical composition is provided in the form of food, beverage, drug, reagent or nutritional supplement.

5. A method for extracting the pharmaceutical composition in claim 1, comprising the steps of:
(a) adding hypotonic buffer solution to organic blue-green algae powder and mixing;
(b) incubating the mixture below room temperature overnight;
(c) separating and purifying the mixture by a centrifuge;
(d) collecting a suspending supernatant and detecting said suspending supernatant by a spectrometer to determine ingredients and content; and
(e) spray drying the supernatant;
characterized in which low-temperature extraction at 0° C.˜18° C. is employed to maintain the bioactivity and nutrients of the pharmaceutical composition.

6. The method according to claim 5, wherein said low-temperature extraction is undertaken under 4° C.
